(19) United States
(12) Patent Application Publication
(10) Pub. No.: US 2008/0022739 A1
(43) Pub. Date: Jan. 31, 2008

(54) Vertical composter with leachate retention system

(76) Inventor: Prakash Aswani, Santa Clara, CA (US)

Correspondence Address: STAINBROOK & STAINBROOK, LLP, 412 AVIATION BOULEVARD, SUITE H, SANTA ROSA, CA, 95403, US

(21) Appl. No.: 11/494,353

(22) Filed: Jul. 26, 2006

(51) Int. Cl. C05F 17/00; C05F 17/02; C12M 1/02

(52) U.S. Cl. 71/6; 435/290.1; 435/290.2

(57) ABSTRACT

A vertical composting apparatus having an upper inlet for solid biodegradable material, at least two, stacked, discrete processing zones for processing the material separated by at least one agitating device separating the upper processing zone and the at least one lower processing zone; a waste inlet above the upper processing zone; a waste outlet below the lowest of the at least one lower processing zones; and a leachate retention system disposed between the upper and lower processings zones for capturing and diverting compost leachate from the upper processing zone back to the upper processing zone for further processing, or to a bio-remediation device, or to a suitable drain or receptable for disposal.

### CROSS REFERENCES TO RELATED APPLICATIONS

The present application is an original regular national filing in the United States.

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

1. Field of the Invention

The present invention relates generally to methods and apparatus for containing, composting, and reducing biodegradable waste, and more particularly to a vertical composting apparatus, and more particularly still to a vertical composting apparatus having discrete, stacked processing zones, and a leachate retention system which prevents pathogen-containing compost leachate from escaping the composting system, and it thus prevents the contamination of ground water and surface water supplies, and further prevents the contamination of finished compost collected in the compost collection zone in the lower portion of the composting apparatus.

2. Discussion of Related Art Including Information Disclosed under 37 CFR §§1.97, 1.98:

It is well known to process and systematically decompose organic waste material using composting apparatus. Under pressure from a burgeoning population with a taste for unrestrained consumption, the development and use of compositing systems and apparatus have evolved and matured rapidly; so too has the understanding of the decomposition process. It is now known that the thermophilic species of aerobic bacteria best suited for breaking down organic waste thrive only in limited ranges of environmental conditions. Those with knowledge in the art understand the importance of controlling several conditions to provide the optimal environment for a rapid decomposition process with a minimum of unpleasant odor. Such conditions include the porosity, temperature, oxygen level, and moisture content of the compost pile and the surrounding atmosphere.

Curiously, the motivation driving the development and use of composting systems, namely the reduction of organic waste, has failed to reduce the waste of physical space required by the compositing systems themselves. Thus, in the commercial setting, it is common to provide large-scale aerated windrow composting systems predicated on a model of moving waste horizontally during the decomposition process. While such systems are suitable for processing large quantities generated by high-volume food industries, restaurants, and cafeterias, or by communities and municipalities that produce unwanted yard trimmings, grease, liquids, and animal byproducts, the process requires frequent turning, hydration, and monitoring. In addition, such systems have a large footprint and require the use of a considerable area of potentially valuable land. Generally, the systems are located in remote, rural, agricultural areas, necessitating the transportation of large amounts of biodegradable waste from urban areas, if such areas are committed to the practice of composting.

It should be noted that the present inventor described and claimed a vertical composting apparatus in International Patent Application Ser. No. PCT/US03/28284, which is incorporated in its entirety herein by reference. This application describes a vertical composting system having a structure closely related to the present inventive composting apparatus. However, it does not solve a problem that remained even after the conception and commercial introduction of that inventive apparatus: namely, suitable handling of compost leachate.

The process of composting large bed volumes of biodegradable material almost invariably results in the production of a byproduct-compost leachate. This liquid is a dark-colored solution that leaches out from the bottom of a compost pile and may contain a high concentration of potentially useful and soluble plant nutrients. However, when leachate is produced in the early stages of composting, before the compost material has been exposed to high temperatures for a suitable period of time, and particularly if the compost bed includes feed stock waste, it will also contain harmful pathogens that can contaminate ground water and surface water supplies. Accordingly, commercially generated leachate byproduct is strictly regulated and allowing the fluid to drain off-site is typically prohibited by law. Rather than disposing of the leachate, however, it may be suitable for further bioremediation through the composting process itself or through exposure to temperatures sufficient to kill pathogens, but not so high as to kill helpful microorganisms.

The known prior art of which the applicant is aware does not disclose, teach, suggest, show, or otherwise render obvious, the invention described and claimed herein. Specifically, no prior art references or commercially available composting systems disclose, show, or include a vertical composting system that rapidly processes large volumes of biodegradable waste, and yet manages, captures, and either processes or properly disposes of composte leachate. There remains a need for such a system, and the present invention provides a solution to the problem of handling compost leachate.

### BRIEF SUMMARY OF THE INVENTION

The present invention is an improved automated in-vessel vertical composting apparatus and method of decomposing waste material that utilizes a vertical arrangement of segregated processing zones for plug-flow processing of biodegradable waste, and it improves known prior art by adding an inventive leachate retention system for preventing pathogen laden compost leachate from escaping the controlled composting system, or even from dripping from upper processing zones into the lower processing zones or collection bins.

It is therefore an object of the present invention to provide a new and improved in-vessel composting apparatus having processing zones in a stackable design, with zones separated by agitators, and with a leachate retention system for preventing the unwanted escape of pathogen-containing composte leachate.

It is another object of the present invention to provide a new and improved composting apparatus that minimizes the amount of land required for a large-scale composting system.

A further object or feature of the present invention is a new and improved composting apparatus that facilitates full control of the environmental conditions for optimal rapid decomposition of biodegradable organic waste and to ensure reliable and consistent output of high quality compost.

An even further object of the present invention is to provide a novel composting apparatus having a plurality of processing zones in a vertical arrangement, thus allowing movement from one processing zone to the next under the influence of gravity alone, and which also employs gravity and the primary force for directing leachate into a fluid collection system.

Accordingly, the composting apparatus of the present invention generally comprises a vertically disposed composting enclosure having at least two physically and functionally discrete processing zones. The zones are separated by either a floor or an agitating device, or both, and each zone preferably includes apparatus for monitoring and controlling the oxygen content, moisture content, temperature of the compost pile. There is also disclosed herein a method of composting biodegradable waste utilizing the inventive apparatus.

Finally, and most essentially, the improved vertical composting system of the present invention includes a leachate retention, collection, and disposal system, disposed below the one or more floor and/or agitation devices separating the processing zones. The leachate handling apparatus comprises a selectively operable gate having a gutter for capturing and diverting leachate into a drain, where it is diverted to a pump which pumps the collected fluid to a disposal container or to apparatus for further bioremediation.

Other novel features which are characteristic of the invention, as to organization and method of operation, together with further objects and advantages thereof will be better understood from the following description considered in connection with the accompanying drawings, in which preferred embodiments of the invention are illustrated by way of example. It is to be expressly understood, however, that the drawings are for illustration and description only and are not intended as a definition of the limits of the invention. The various features of novelty that characterize the invention are pointed out with particularity in the claims annexed to and forming part of this disclosure. The invention does not reside in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE SEVERAL VIEWS OF THE DRAWINGS

The invention will be better understood and objects other than those set forth above will become apparent when consideration is given to the following detailed description thereof. Such description makes reference to the annexed drawings wherein:

FIG. 1 is an upper front left perspective view of a first preferred embodiment of the vertical composting apparatus of the present invention;

FIG. 2 is an upper rear left perspective view thereof;

FIG. 3 is a top plan view thereof;

FIG. 4 is a front side view in elevation thereof;

FIG. 5 is a cross-sectional right side view in elevation thereof, taken along section line 5 of FIG. 1, and showing the leachate retention gate in the up position;

FIG. 6 is a cross-sectional rear view in elevation thereof, taken along section line 6 of FIG. 2, and showing the leachate retention gate in the up position;

FIG. 7 is the same cross-sectional rear view in elevation as FIG. 6, but showing the leachate retention gate in the down position;

FIG. 8 is an enlarged detail side view taken along line 8 of FIG. 7, showing the structural and operational elements of the leachate retention gate of the first preferred embodiment of the present invention;

FIG. 9 is an upper left front perspective view of a second preferred embodiment of the inventive vertical composter with leachate retention system;

FIG. 10 is an upper rear left view thereof;

FIG. 11 is cross-sectional right side view in elevation thereof, taken along section line 11 of FIG. 9, showing the leachate retention gate in the closed position;

FIG. 12 is a cross-sectional rear view in elevation thereof, taken along section line 12, showing the leachate retention gate in the close position;

FIG. 13 is the same cross-sectional rear view in elevation as FIG. 12, but showing the leachate retention gate in the open position;

FIG. 14 is an enlarged detail view showing the structural and operational elements of the leachage retention gate of the second preferred embodiment, taken along detail line 14 of FIG. 13;

FIG. 15 is partial cross-sectional upper left perspective view showing a third preferred embodiment of the vertical composter with leachate retention system of the present invention;

FIG. 16 is a cross-sectional rear view in elevation thereof, taken along section line 16 of FIG. 15, showing the leachate retention gate of the third preferred embodiment in the closed position;

FIG. 17 is the same cross-sectional rear view in elevation as FIG. 17, but showing the leachate retention gate in the open position;

FIG. 18 is an enlarged detail view taken along detail line 18 of FIG. 17 showing the leachate retention gate of the third preferred embodiment of the inventive vertical composter; and

FIG. 19 is a schematic cross-sectional side view in elevation showing a fourth preferred embodiment of the present invention, as well as its use in a large scale composting system.

### REFERENCE NUMERAL LEGEND

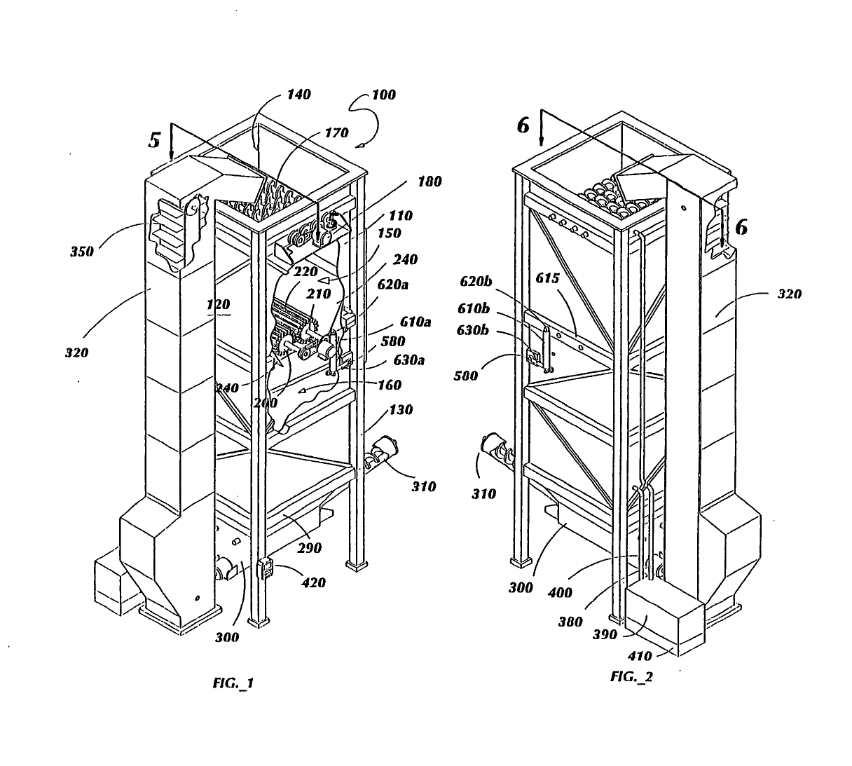

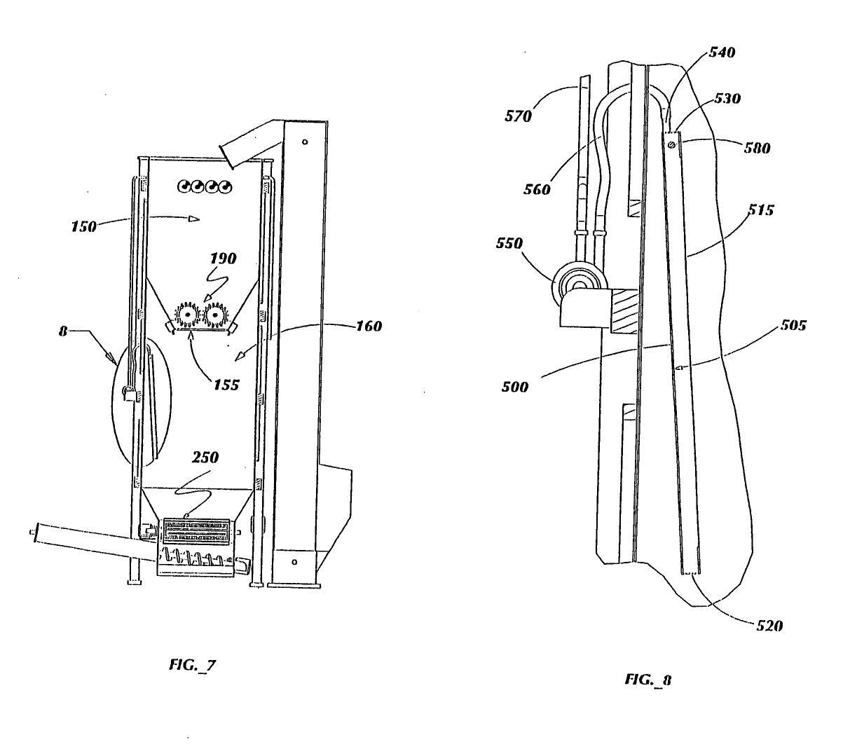

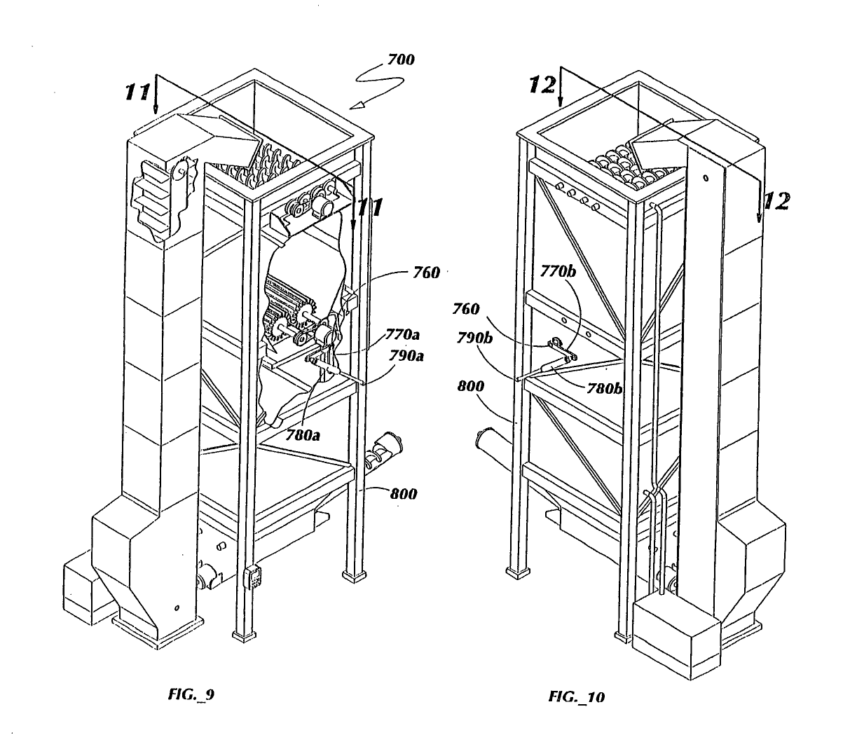

### FIGS. 1-8

100 vertical composting system (first preferred embodiment)
110 vessel housing
120 housing wall
130 frame
140 upper waste inlet
150 upper processing zone
155 processing zone passage
160 lower processing zone
170 leveling means/auger screw(s)
180 motor
190 upper agitation device
200 first upper tumble roll feeder
210 second upper tumble roll feeder
220 roller paddles
230 motor
240 baffle plates
250 lower agitation device
260 first lower roller
270 second lower roller
280 motor
290 inwardly tapering walls
300 discharge outlet
310 discharge conveyor system/screw auger
320 waste input conveyor system/vertically disposed belt conveyor
330 waste loading inlet
340 vertically disposed tube
350 motor driven bucket conveyor cable
360 material outlet
370 misting nozzles
380 gas outlet duct
390 bio-filter
400 air inlet
410 air heating and cooling unit
420 programmable logic means/PLC 500 or computer
500 leachate retention gate (first preferred embodiment)
505 bottom of gate
510 side of gate
515 side of gate
520 outboard gutter
530 inboard gutter
540 drain
550 pump
560 pump inlet hose
570 pump outlet hose
580 axle
590 open gate position
600 closed gate position
610a hydraulic cylinder
610b hydraulic cylinder
615 horizontal rail of housing frame
620a pivot point
620b pivot point
630a lever arm
630b lever arm

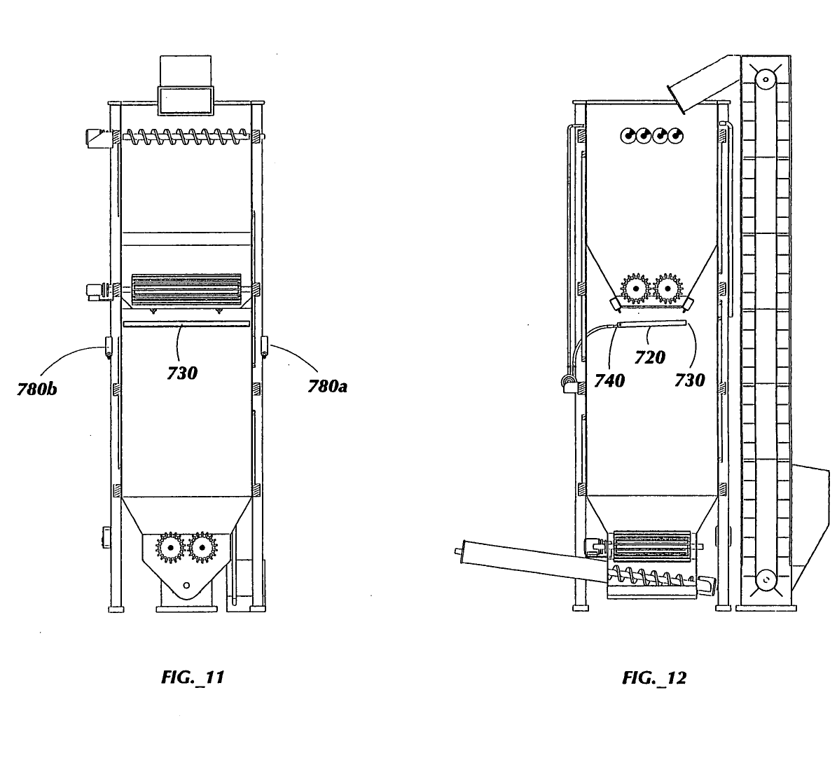

### FIGS. 9-14

700 second preferred embodiment of the present invention
710 leachate retention system
720 leachate gate bottom
730 outboard gutter
740 inboard gutter
750 passageway between baffles
760 axle
770a lever arm
770b lever arm
780a hydraulic cylinder
780b hydraulic cylinders
790a pivot point
790b pivot point
800 vertical support member

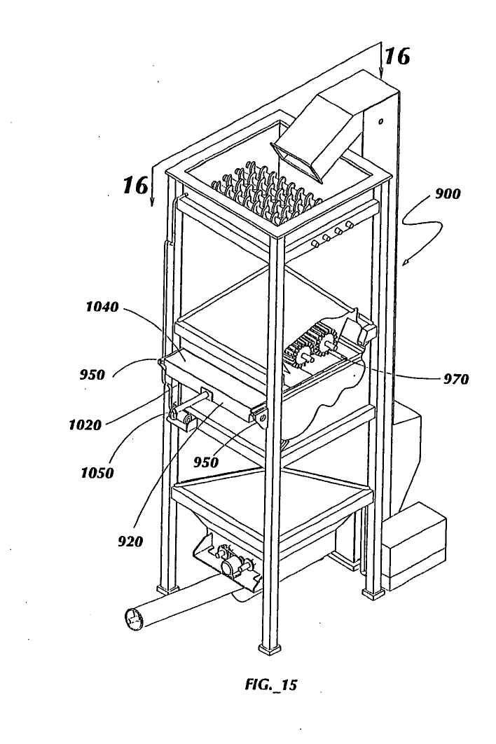

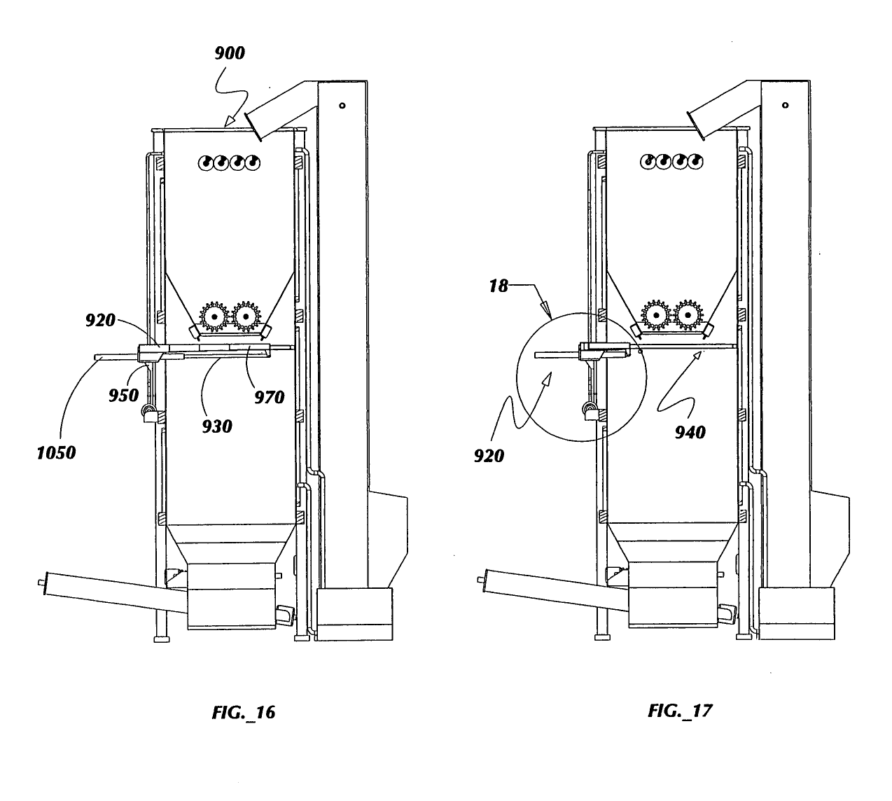

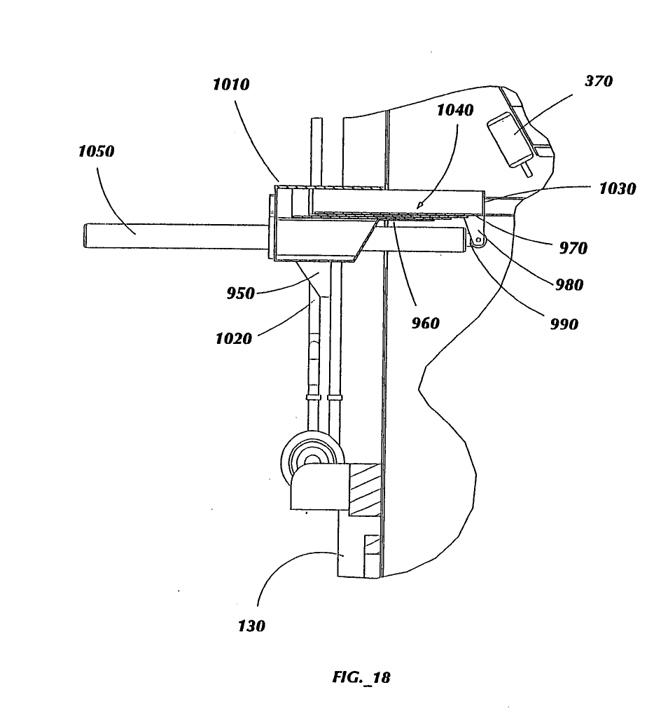

### FIGS. 15-18

900 third preferred embodiment of the present invention
910 leachate retention system
920 telescoping slide gate
930 fully extended (gate closed) configuration
940 collapsed (gate open) configuration
950 mounting brackets
960 telescoping members
970 outermost telescoping member
980 rod connection bracket
990 cylinder rod
1010 fixed base member
1020 drain
1030 outermost telescoping member turned up end
1040 pocket
1050 hydraulic cylinder

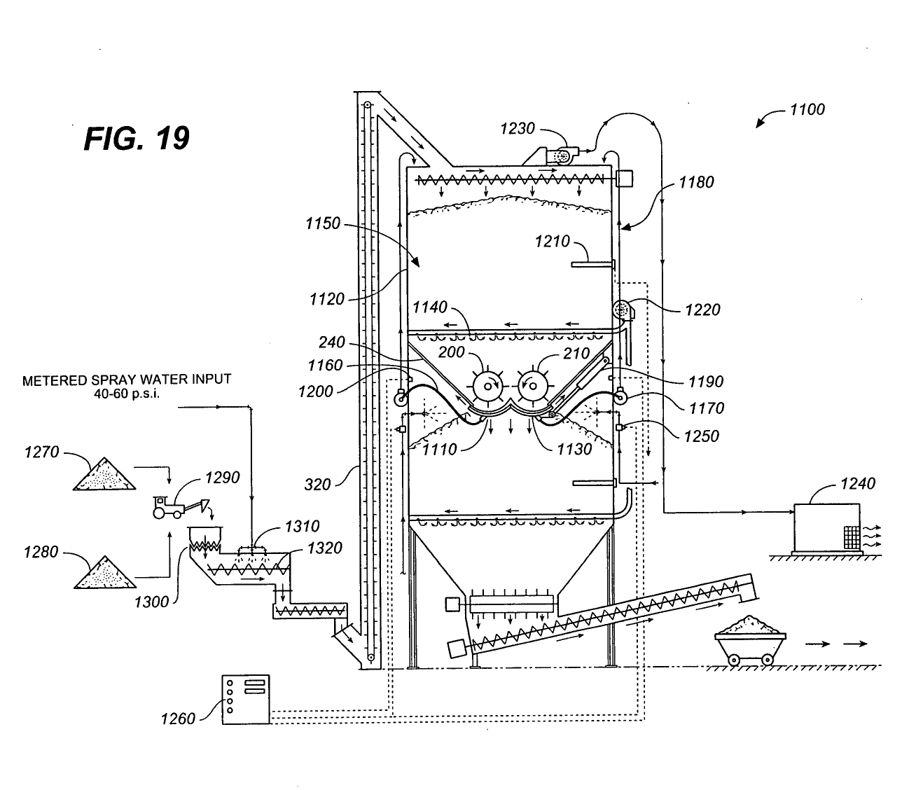

### FIG. 19

1100 fourth preferred embodiment
1110 clam shell gate
1120 housing walls
1130 low point of clam shell halves
1140 first compost volume
1150 upper composting zone
1160 flexible hose
1170 liquid sludge pump
1180 waste water return pipe
1190 hydraulic cylinders
1200 load cells
1210 temperature sensors
1220 air cooling system blowers
1230 air discharge blower
1240 bio-filter
1250 misting valves
1260 control system and monitor
1270 carbon based materials
1280 pre-sorted biodegradable waste
1290 material moving and loading equipment
1300 shreader/mixer
1310 metered water spray
1320 screw conveyor

### DETAILED DESCRIPTION OF THE INVENTION

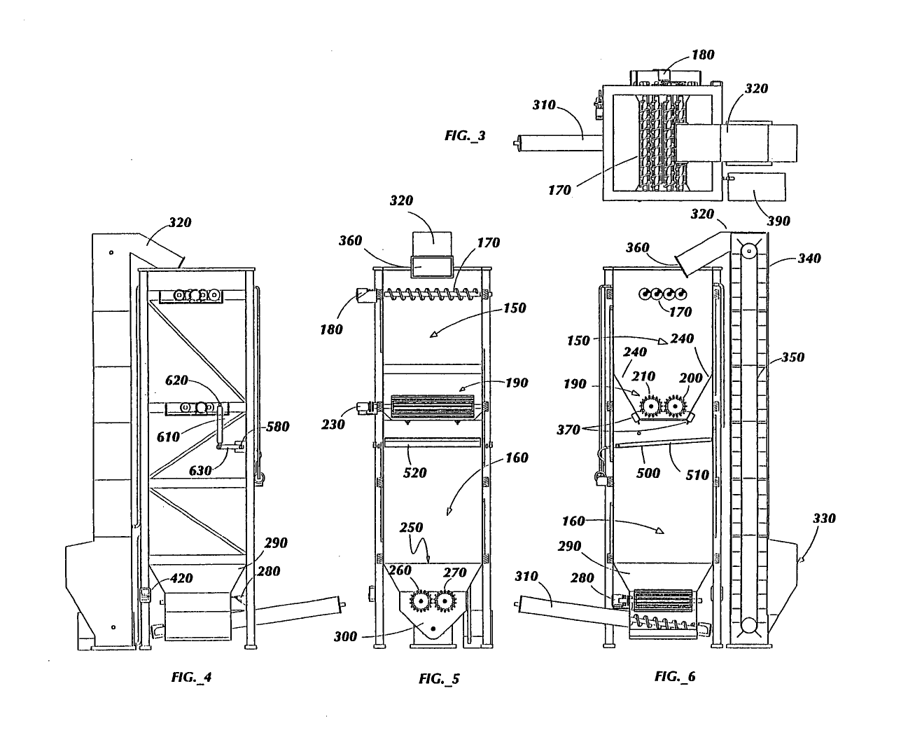

Referring to FIGS. 1 through 19, wherein like reference numerals refer to like components in the various views, FIG. 1 is an upper front left perspective view of the first preferred embodiment of the present invention, while FIGS. 2, 3, and 4 are, respectively, an upper rear left perspective view thereof, a top plan view thereof, and a front side view in elevation thereof. FIGS. 5-7 are cross-sectional views showing leachate retention gate in the up and down positions, while FIG. 8 is a detail side view of the leachate retention gate of the first preferred embodiment.

Collectively, these views show the inventive apparatus 100 comprises a vessel housing 110 having at least one housing wall 120. For purposes of composting waste according to the present invention, the vessel is preferably either generally cylindrical, in which event the wall is singular and contiguous, or cuboid, in which event the housing has four walls comprising sides. However, in the first preferred embodiment the vessel housing is cuboid and is supported on a frame 130. Vessel dimensions will vary according to the scale of the composting operation, though heights ranging from approximately nine to eighteen meters (thirty to sixty feet) have proven most serviceable and work best for materials processed for a typical resonance time in a plug-flow system.

A waste inlet 140 is located at the top of the housing above an upper processing zone 150. The inlet may be either directly above the upper processing zone or to the side, depending upon the means provided for distributing and spreading the waste before introduction into the first processing zone. Positioned below the first processing zone is a lower processing zone 160, and the two zones are brought into communication with one another through a processing zone passageway 155.

Waste introduced into the housing through the waste inlet is leveled and evenly distributed over the upper processing zone by leveling means 170, preferably comprising at least one auger screw having either continuous solid or ribbon flights. It will be appreciated, however, that numerous suitable alternative means may be employed, including, for instance, a rotary leveler. The at least one auger screw is driven by a reversible motor 180, which permits periodic and systematic movement of the waste material in each direction along the length of the auger screw.

At least one agitating device 190 is interposed between the upper processing zone 150 and the lower processing zone 160. Preferably, the agitating device comprises a first set of side-by-side tumble roll feeder 200, 210, each of which include a plurality of radially disposed surface paddles or bars 220. The tumble roll feeders are actuated by one or more reversible motors, 230. When operated, the rollers are preferably rotated in opposite directions such that when viewed from either end, the left hand roller rotates clockwise and toward the right hand roller, and the right hand roller rotates counterclockwise and toward the left hand roller. However, each tumble roll feeder may also be rotated in the opposite direction, as long as both rollers are not simultaneously rotated in the same direction.

In alternative embodiments, the agitating device may be replaced by a different, non-agitating device such as a stationary grid, bars, a moving floor, slide gate, or a hatch. In these embodiments, the device, which separates the different zones in the apparatus, keeps the weight of compost material in the upper region of the composting apparatus from compressing compositing material at the lower region of the composting apparatus. The fundamental concept of processing the compost in vertically disposed but discrete processing zones is nonetheless preserved in such embodiments.

In the preferred embodiments of the present in invention, a lower agitation device 250 is positioned at the base of the lower processing zone 160. Preferably the agitation device comprises a second set of side-by-side rollers 260, 270 that operate in the manner as described above. The second set of rollers is actuated by at least one second motor 280.

Throughout the composting cycle, the agitation means are operated at predetermined intervals. The agitators mix, delump, aerate, and turn over the compost material inside vessel. The agitation means also support the compost material above them so that the full weight of compost pile does not compact the compost material below it, thereby limiting oxygen supply to material deep within the pile. Effective agitation can be enhanced by the provision of one or more baffle plates 240 positioned on the interior surface(s) of the vessel housing, which direct the bulk volume into the agitator paddles or rollers. Alternatively, the vessel housing itself include inwardly tapering walls 290, in the fashion of a hopper, to direct the material flow into the agitators and through the passageway at the lower portion of the processing zone.

A discharge outlet 300 is positioned directly below the lower agitation device. This portion of the vessel housing is configured to direct finished compost into a discharge conveyor system 310, preferably a screw auger, which transports nutrient rich plant growth media from the apparatus to holding bins or other containers to await use.

For large scale processing of biodegradable waste, it is preferable for the inventive system to include a waste input conveyor system 320, preferably a vertically disposed bucket conveyor. The preference of conveyor type is driven by the angle or orientation of the system, and the orientation advances the space-saving purpose of the inventive apparatus, though it is not critical to its effective function. Thus, alternative systems could be employed if the conveyor were more horizontally disposed. The waste input conveyor system includes a waste loading inlet 330, a vertically disposed tube 340, a motor driven bucket conveyor cable 350, and a material outlet 360, all of which are well known in the art.

Misting nozzles 370 may be positioned in any processing zone to accomplish several purposes, including: increasing atmospheric humidity; increasing the water content of the compost pile; and washing leachate residue from the upper portions of the leachate retention gate (described and discussed in detail below). Optimally added water will not be required in the upper processing zone.

Environmental control systems may be provided to tightly regulate the conditions under which the material is processed during the resonance period. As noted, misting nozzles can be provided to add water to selected processing zones. Additionally, temperature, oxygen level, and moisture level sensors may be installed in one or more of the processing zones so that adverse conditions can be corrected promptly. The sensors are not shown as they are common and well known in the art. The system may further include gas outlet duct 380 for venting and removing exhaust gasses produced by the composting process. The air removed from the system may be processed in a bio-filter 390, released directly into the atmosphere (if appropriate and permitted), or directed into a ground berm. Either fresh or recycled air may be directed into the vessel housing via air inlet 400, and such may be heated or cooled by a heating and cooling unit 410, preferably co-located with the bio-filter. All of the environmental systems are preferably coordinated with one another, as well as with the agitation systems, through programmable logic means, either a programmable logic controller or a computer 420.

The essential improvement provided by the present inventive system and apparatus is a novel compost leachate retention system. This novel system is illustrated in all of the figures, which collectively show four preferred embodiments. The first embodiment is illustrated in FIGS. 1-8. These views show a leachate retention gate 500 disposed immediately underneath the upper agitator and between the upper and lower processing zones. The gate is substantially square or rectangular (depending on the vessel housing shape) and acts as a catch basin for leachate dropping from the upper processing zone or zones. It includes a bottom 505 with two turned up sides 510, 515, and further includes at least one, and preferably two, gutters, including an outboard gutter 520, and an inboard gutter 530, which comprise the first and second ends, respectively of the leachate gate. The inboard gutter functions as a drain side gutter. The gate is preferrably substantially square or rectangular (depending on the vessel housing shape) and the perimeter dimensions of the contiguous exterior sides closely conform to the interior dimensions of the vessel housing, with tight clearances sufficient to be suitable for capturing all of the leachate dripping down through the processing zone passage yet large enough to allow the gate to swing unimpeded both downwardly and upwardly during operation.

The inboard (drain side) gutter 530 includes a drain 540 in fluid communication with a pump 550 via a pump inlet hose 560. The pump sends collected leachate fluid through a pump outlet hose 570 for further processing in the compost apparatus, or otherwise directs the collected liquids to a receptacle or dedicated drain for proper disposal or bio-remediation.

Next, the leachate gate is pivotally connected through an axle 580 to mechanical means for swinging the door downwardly into a open position 590, so as to permit the compost material to pass from the upper processing zone into the lower processing zone, and then to raise the door back into a closed position 600 for capturing and draining compost leachate. Preferably the mechanical means is at least one, and preferably two, linear actuators, such as hydraulic cylinders 610a, 610b, powered by a hydraulic system (not shown), as is well known in the art. The cylinders are pivotally attached to horizontal rails 615 of the housing frame at pivot points 620a, 620b, and drives lever arms 630a, 630b, each rigidly coupled to the rotatable axle 580.

It will be appreciated that the leachate gate is preferably angled slightly downwardly toward the drain side gutter when in the closed position so that leachate, either diluted or undiluted, and other fluids do not cause the gate to overflow and so as to provide for continuous drainage from the catch basin.

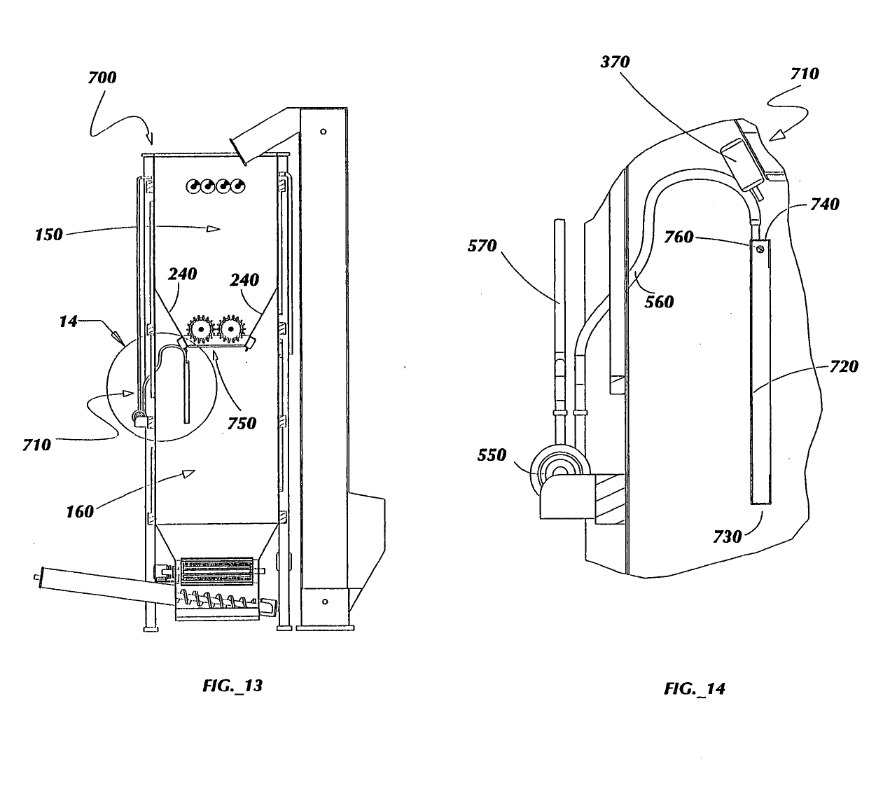

Referring now to FIGS. 9-14, in a second preferred embodiment 700 of the present invention, the leachate retention system 710 is identical in virtually every respect to that of the leachate retention system of the first preferred embodiment, except that the width dimension of the leachate gate bottom 720 from its outboard gutter 730 to its inboard gutter 740 is sized just sufficiently to cover the dimensions of the passageway 750 between baffles 240. In this manner, it captures all of the leachate and spray from nozzles 370, while minimizing the extent to which it swings into the lower processing zone when opened (FIG. 13). This allows the lower processing zone to have a lower height and/or to reduce the frequency with which the finished compost material collected must be dumped.

The reduced size of the leachate gate in the second preferred embodiment necessitates a repositioning of the axle 760 through which the leachate gate is rigidly connected to first and second lever arms 770a, b, which are pivotally connected to first and second hydraulic cylinders 780a, 770b, which are in turn pivotally connected at pivot points 790a, 790b to vertical support members 800 of the housing frame.

In a third preferred embodiment of the present invention 900, the leachate retention system 910 employs a telescoping slide gate 920 rather than a swinging gate. FIG. 16 shows its fully extended (gate closed) configuration 930 while FIG. 17 shows the gate in a collapsed (gate open) configuration 940. Use of such apparatus further reduces the space required by the vertical composing housing by eliminating all downswing of the retention system gate.

The telescoping slide gate is mounted on brackets 950 installed on and affixed to the housing frame 130. The gate itself includes a plurality of telescoping members 960, including an outermost telescoping member 970 having a cylinder connection bracket 980 for connection of the cylinder rod 990, and further including a fixed base member 1010, which includes a drain 1020. All of the telescoping members have turned up sides, and the outermost telescoping member has a turned up end 1030. Further, the fixed base member includes a pocket 1040 into which all telescoping members nest when the gate is collapsed. However, an outboard gutter is not required in this embodiment inasmuch as the gate is not swung either upwardly or downwardly during operation. However, the gate is extended, it is angled slightly from its outermost telescoping member downwardly to the innermost telescoping member so that liquids captured by the gate naturally run down to the drain in the innermost telescoping member.

The hydraulic cylinder 1030 may be a single-acting external return linear actuator with piston and rod travel sufficient to extend the telescoping gate underneath the passageway between the baffles. In such a case, the cylinder may be installed at the mounted end of the gate and will extend a short distance outside the housing frame. Alternatively, the hydraulic cylinder can itself be a telescoping cylinder which collapses substantially in coordination with the collapsing gate, and thus need not extend significantly outside the vertical composter housing.

A fourth preferred embodiment of the present invention is shown in FIG. 19, which is a schematic cross-sectional side view in elevation. This view illustrates a vertical composter 1100 which, once again, includes a leachate retention system, but in this embodiment, the gate disposed below the baffle plates 240 and tumble roll feeders 200/210 is a clam shell gate 1110 having two halves, each pivotally connected to the baffle plates and/or the composter housing walls 1120. Each half is essentially an arcuate panel having a trough portion with a low point 1130 disposed in the generally central portion of the trough and a hole through the low point forming a drain. Liquid from the first compost volume 1140 in the upper composting zone 1150 is drawn through the drain and a flexible hose 1160 connected to the drain by a liquid sludge pump 1170, which preferably includes a macerator. Thereafter, it is pumped back into the upper processing zone through a waste water return pipe 1180 or elsewhere for further processing.

As with the earlier embodiments, the fourth preferred embodiment includes actuators, preferably hydraulic cylinders 1190, which selectively open and close the clam shell halves. Other notable features of the inventive system include load cells 1200 for batch weighing compost volumes, temperature sensors 1210, one or more air cooling system blowers 1220, for introducing air into the composting zones, an air discharge blower 1230 for discharging clean air from the system, possibly through a bio-filter 1240, and misting valves 1250 for controlling the introduction of water spray into the processing zones. All of these environmental controls are under the control of a control system and monitor 1260, to which they are connected.

As FIG. 19 shows, a system utilizing the apparatus of the present invention facilitates the processing of large volumes of carbon based materials 1270, such as wood, chips, paper, and the like, as well as pre-sorted biodegradable waste 1280. Material moving and loading equipment 1290 may be employed to load the materials into a shreader and mixer 1300, where it may be optimally hydrated for composting with metered water spray 1310. From there it is conveyed via a screw conveyor 1320 to the bucket elevator 320.

Method Steps: Referring now especially to FIG. 19, the following steps comprise the essential processing sequence in employing the vertical composting apparatus of the present invention:

(1) Organic waste (biodegradable waste) is combined with a bulking agent (wood chips, sawdust, etc.) to achieve a proper carbon-to-nitrogen ratio and a correct porosity in waste material mixture.

(2) The organic waste and bulking agent are mixed thoroughly (typically in a automated four auger mixer, though this can also be accomplished with a front loader or an equivalent method).

(3) The mixed organic waste and bulking agent are loaded into a bucket elevator receiving hopper. This is typically accomplished with a conveyer, though it can also be done with a front loader or an equivalent method.

(4) The waste mixture is vertically conveyed by bucket elevator to the top of the composting apparatus.

(5) The waste mixture is deposited into the top of the composting apparatus.

(6) The waste mixture is distributed evenly in the horizontal plane by leveling screws into the upper processing zone in the composting apparatus.

(7) The waste mixture is retained in the upper processing zone for approximately seven days. Each day the waste mixture is moving down as compost is unloaded and new waste is added).

(8) The waste mixture reaches the agitation system rollers on approximately the seventh day.

(9) The waste mixture is turned, agitated, de-lumped and forced into the lower processing zone by the agitation system rollers.

(10) Water is added to the waste mixture if needed to maintain adequate moisture content after the waste mixture passes through the agitation system rollers.

(11) The waste mixture is retained in the lower processing zone for approximately seven additional days. Each day the waste mixture progressively moves down as compost is unloaded and new waste is added.

(12) After approximately the fourteenth day, retention in the composting apparatus waste mixture has been stabilized to meet pathogen destruction and vector attraction regulations and now comprises a nutrient rich plant growth media.

(13) Plant growth media is discharged from the bottom of the composting apparatus via screw conveyor or an equivalent method for further post processing (such as curing, drying, screening, bagging, etc.). It is loaded into transportation means which convey the processed compost to its destination for utilization.

Monitoring/Controls: The following controls and monitoring systems may be employed throughout the composting cycle to ensure the maintenance of optimum composting conditions:

(1) Vertically oriented temperature probes may be permanently fixed in the center of the composting mass to give continuous feedback to the control panel which activates fans or blowers to effectively regulate temperature and oxygen within the composting mass. The temperature may be controlled so as not to exceed 71° C. (160 degrees Fahrenheit) and not to go below 54° C. (131 degrees Fahrenheit). Positive air may be pushed by blowers into the bottom of the composting mass and negative air pressure is exerted on top of the composting mass to pull air from the top of the composting mass to a bio-filter. The bio-filter may include a specific mixture of stabilized mature compost, woodchips and other filtering media to clean exhaust air in order to prevent odor from being emitted into the surrounding environment. Other air filtration devices can be utilized to scrub or clean the air instead of a bio-filter.

(2) Moisture sensors can be positioned throughout the composting mass to give continuous feedback to the control panel which activates irrigation emitters to control moisture content of the composting material. The moisture level of the composting mass can be maintained at approximately 60% to optimize the composting process.

(3) Throughout the composting cycle, all compost leachate generated is captured, contained, and diverted for appropriate handling by the leachate retention system. Leachate is recycled and used for irrigation purposes or can be diverted to a drain or container for appropriate disposal. This control step is the heart of the present inventive system.

The foregoing disclosure is sufficient to enable one having skill in the art to practice the invention without undue experimentation, and provides the best mode of practicing the invention presently contemplated by the inventor. While there is provided herein a full and complete disclosure of the preferred embodiments of this invention, it is not intended to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Accordingly, the proper scope of the present invention should be determined only by the broadest interpretation of the appended claims so as to encompass all such modifications as well as all relationships equivalent to those illustrated in the drawings and described in the specification.

What is claimed as invention is:

1. A composting apparatus comprising:
a housing including a housing frame and a housing wall;
an upper processing zone in said housing;
at least one lower processing zone positioned below said first processing zone;
a barrier separating said upper processing zone from said second processing zone so as to prevent particulate material in said upper processing zone from passing into said lower processing zone;
a biodegradable waste inlet above said upper processing zone;
a finished composte outlet below said at least one lower processing zone; and
liquid barrier means for preventing compost leachate from passing from said upper processing zone to said lower processing zone.

2. The composting apparatus of claim 1, wherein said barrier includes at least one baffle angled inwardly from said side of said housing in said upper processing zone so as to define a passage between said upper processing zone and said lower processing zone.

3. The composting apparatus of claim 2, wherein said barrier further includes at least one agitator disposed in the passage defined by said at least one baffle.

4. The composting apparatus of claim 4, wherein said liquid barrier means is a leachate retention gate disposed immediately below the passage defined by said at least one baffle, and wherein said leachate retention gate includes a drain.

5. The composting apparatus of claim 4, wherein said leachate retention gate is pivotally connected through an axle to mechanical means for swinging said gate downwardly into a open position, so as to permit compost material in said upper processing zone to pass from said upper processing zone into said lower processing zone, and then to raise the door back into a closed position for capturing and draining compost leachate.

6. The composting apparatus of claim 5, wherein said leachate retention gate has an outboard gutter and an inboard gutter, and wherein said drain is disposed in said inboard gutter.

7. The composting apparatus of claim 6, wherein said leachate retention gate has contiguous exterior sides generally conforming to the interior dimensions of said wall of said vessel housing, and clearances sufficiently tight to capture substantially all of the leachate dripping down through the passage while large enough to allow said leachate rentention gate to swing both downwardly and upwardly during operation.

8. The composting apparatus of claim 7, wherein said mechanical means is at least one linear actuator.

9. The composting apparatus of claim 8, wherein said leachate gate is angled when in the closed position.

10. The composting apparatus of claim 4, wherein said leachate retention gate is sized to cover the passageway defined by said at least one baffles.

11. The composting apparatus of claim 4, wherein said leachate retention gate is a telescoping slide gate mounted on said housing frame.

12. The composting apparatus of claim 11, further including a linear actuator operatively connected to said leachate retention gate.

13. The composting apparatus of claim 4, wherein said leachate retention gate is a clam shell gate having two halves which close together immediately below said passage.

14. A method for composting material comprising:
(a) providing a vertical composting system having a vertically oriented housing including a wall, an upper processing zone in the housing, at least one lower processing zone positioned below the first processing zone, at least one device separating the upper processing zone from said second processing zone; a waste inlet above the upper processing zone, a finished composte outlet below the lower processing zone, a temperature and moisture control system, and a leachate retention system;
(b) introducing a biodegradable material through the waste inlet;
(c) processing the biodegradable material in the upper processing zone while maintaining optimum processing conditions of moisture and temperature;
(d) capturing and diverting any liquid leachate produced in the upper processing zone with the leachate retention system;
(e) moving at least one agitating device below the upper processing zone thereby allowing the processed biodegradable material to pass into the lower processing zone;
(f) processing the compostable material in the lower processing zone to form finished compost material suitable for use in agriculture and horticulture; and
(g) removing the composted material through an outlet below the lower processing zone.

15. The method of claim 14, wherein step (d) comprises diverting the captured leachate back to the upper processing zone for further processing.

16. The method of claim 14, wherein step (e) comprises diverting the captured leachate to a receptable for shipment to a disposal site or to other apparatus for bioremediation of the leachate.

17. The method of claim 14, wherein step (e) comprises diverting the captured leachate to a drain for disposal.
